(19) United States
(12) Patent Application Publication
(10) Pub. No.: US 2026/0217773 A1
(43) Pub. Date: Jul. 30, 2026

(54) COMPOSITIONS AND METHODS FOR TREATING AND PREVENTING HEADACHES

(71) Applicant: NEUROCARRUS INC., Monterey, CA (US)

(72) Inventors: Rylie McDONELL, Monterey, CA (US); Derek ALLEN, Monterey, CA (US); Paul BLUM, Monterey, CA (US)

(73) Assignee: NEUROCARRUS INC., Monterey, CA (US)

(21) Appl. No.: 19/331,900

(22) Filed: Sep. 17, 2025

Related U.S. Application Data
(60) Provisional application No. 63/695,737, filed on Sep. 17, 2024.
(60) Provisional application No. 63/867,540, filed on Aug. 20, 2025.

(51) Int. Cl. C07K 14/33; A61K 38/00; A61K 45/06; A61K 47/64; A61P 25/06

(52) U.S. Cl. CPC C07K 14/33; A61K 45/06; A61K 47/6415; A61P 25/06; A61K 38/00

(57) ABSTRACT

Compositions and methods for preventing or reducing the occurrence of headache disorders in a subject in need thereof comprising administering to the subject an effective amount of a pharmaceutical composition comprising a nontoxic engineered protein derived from a botulinum toxin complex. The headache disorder may be selected from episodic migraine headaches, tension-type headaches, cluster headaches, hemiplegic migraine headaches, or retinal migraine headaches.

### REFERENCE TO RELATED APPLICATIONS

This application claims benefit to and priority to U.S. Provisional Application Ser. No. 63/695,737, filed on Sep. 17, 2024, and to U.S. Provisional Application Ser. No. 63/867,540, filed on Aug. 20, 2025, which are hereby incorporated by this reference in their entirety.

### SEQUENCE LISTING

The application contains a Sequence Listing which has been submitted electronically in .XML format and is hereby incorporated by reference in its entirety. Said .XML copy, created on Oct. 17, 2025, is named “034809.004US1.xml” and is 4,121 bytes in size. The sequence listing contained in this .XML file is part of the specification and is hereby incorporated by reference herein in its entirety.

### FIELD

This invention relates generally to compositions and methods of treating headache disorders in a subject in need thereof, comprising administering to the subject an effective amount of a pharmaceutical composition comprising a nontoxic engineered protein derived from a botulinum toxin complex.

### BACKGROUND

Migraines are complex neurological disorders with a multifaceted mechanism of action. They pose a large burden on the global healthcare system as the third most common medical condition in the world (Vos T et al., Lancet. 2012 Dec. 15; 380(9859):2163-96.). The pathophysiology of migraines involves the modulation of pain-producing structures in the brain by the central and peripheral nervous systems (Goadsby, P. J. (2005), Headache: The Journal of Head and Face Pain, 45: S14-S24). One aspect is cortical spreading depression (CSD), a depolarization of neuronal and glial cell membranes that spreads over the brain cortex and is implicated as a cause of migraine aura (Liem M K et al. Cephalalgia. 2010; 30(11):1284-1289). The neuropeptide calcitonin gene-related peptide (CGRP) plays a crucial role in migraines, with animal studies showing its direct causation of migraine behavior. However, given CGRP's role in multiple neuronal functions, the direct link in humans remains unclear (Russo A F. Annu Rev Pharmacol Toxicol. 2015; 55:533-52).

Triptans, such as sumatriptan, act on serotonin receptors in the brain to alleviate migraine symptoms by modulating nociceptive thresholds (Ahn A H, Basbaum A I. Pain. 2005 May; 115(1-2):1-4). Furthermore, the combination of sumatriptan and naproxen has been shown to provide quick pain relief based on sumatriptan's mechanism of action and sustained response due to naproxen's action (Blumenfeld A M, et al. Headache. 2013 April; 53(4):644-55). But the use of triptans and other traditional, acute migraine therapeutics is limited to the relatively short duration of activity at 4-6 hours and the limited recommended use at days in a month (Glynos N G, et al. bioRxiv [Preprint]. 2024 Apr. 22:2024.04.19.589047; Pringsheim T, Becker W J. BMJ. 2014 Apr. 7; 348:g2285).

Functional neuroimaging studies have identified key areas in the central nervous system involved in the early phases of a migraine attack, shedding light on the underlying mechanisms of the disorder (Recober A. Continuum (Minneap Minn). 2021 Jun. 1; 27(3):586-596). It has also been found that the cerebral cortex plays pivotal roles in initiating, propagating, and terminating migraine attacks, as well as during the interictal phase (Dai W, et al. Mol Pain. 2021 January-December; 17:17448069211050246). The hypothalamus also shows increased activation and connectivity in chronic migraine, indicating its involvement in disease progression (Robblee J, et al. Headache. 2024; 64: 869-872).

Chronic migraines have a prevalence of about 2% in the United States (Ashina M, et al. Migraine: epidemiology and systems of care. Lancet. 2021 Apr. 17; 397(10283):1485-1495). Chronic migraines are characterized by 15 or more headache days per month, with at least 8 days showing typical migraine features (Torres-Ferrns M, et al. J Headache Pain. 2020 Apr. 29; 21(1):42). 11% of the global population experiences migraines, with 3% suffering from chronic daily headaches (Stovner Lj, et al. Cephalalgia. 2007 March; 27(3):193-210). The Chronic Migraine Epidemiology and Outcomes Study found that disability was more than threefold greater in the chronic migraine cohort compared to the episodic migraine cohort (Nye B L, Headache. 2015 March; 55(3):359-80). Research indicates that chronic migraine is more prevalent in certain demographic groups, for example migraines are three times more common in women than men, (Larkin L. Menopause. 2022 May 1; 29(5):606-608).

Preventive migraine therapy is crucial for reducing the frequency and severity of migraine attacks. Current preventive treatments for chronic migraines include topiramate and onabotulinumtoxin A (Agostoni E C, et al. J Headache Pain. 2019 Aug. 30; 20(1): 92). Additionally, off-label medications like propranolol, timolol, topiramate, and divalproex sodium are approved by the FDA for migraine reduction (Simonetta I, et al. Int J Mol Sci. 2022 Mar. 11; 23(6):3018). But these traditional classes of preventives recommended in clinical guidelines have shown a limited effectiveness (Gottschalk C, et al. Ther Adv Neurol Disord. 2022 May 31; 15:17562864221095902). Monoclonal antibodies targeting the calcitonin gene-related peptide (CGRP) and receptor pathway, such as erenumab, fremanezumab, and galcanezumab, have also been approved for migraine prevention (Yuan H, et al. Headache. 2019 July; 59 Suppl 2:20-32). Newer treatments like CGRP-targeting monoclonal antibodies have shown strong efficacy, but they are not suitable for all patients due to factors such as cost, tolerability, and individual response variability (Yuan et al., 2019). While there are several preventive therapies available for migraines, each with its set of limitations, there remains a need for treatment options aimed at addressing these challenges and providing more effective and well-tolerated solutions for individuals suffering from migraines.

Preclinical animal models for migraines have been developed upon the known components that initiate migraine symptoms in people. One example is the CGRP-induced migraine pain model demonstrated in rodents (Avona, A. et al. Journal of Neuroscience 29 May 2019, 39 (22) 4323-4331). Following a systemic injection of the neurotransmitter CGRP, rats and mice have demonstrated a heightened amount of migraine behaviors, including light sensitivity, reduction in wheel usage, periorbital hypersensitivity, and changes in open field behavior (Wattiez A S, et al. Expert Opin Ther Targets. 2020 February; 24(2):91-100). Pre-treatment with monoclonal antibodies for both the peptide and receptor for CGRP has been shown to reduce the migraine state for these animals (Mason B N, et al. J Neurosci. 2017 Jan. 4; 37(1):204-216). In addition, the use of triptan drugs has been shown to relieve migraine symptoms when administered after the onset of migraine behaviors (Asghar M S, et al. Ann Neurol. 2011 April; 69(4):635-45).

Thus, migraine sufferers have an urgent medical need for more effective and/or tolerable treatment options.

Provided herein are methods of treating headache disorders in a subject in need thereof comprising administering to the subject an effective amount of a pharmaceutical composition comprising a nontoxic engineered protein derived from a botulinum toxin complex, also referred to as NL-17.

### SUMMARY

In accordance with the purpose(s) of this invention, as embodied and broadly described herein, this invention, in one aspect, relates to a composition comprising: a) a target cell binding unit; b) a pore-forming unit, c) a rigid synthetic linker linking the target cell binding unit and the pore-forming unit, and d) a payload unit comprising the agent, wherein said payload unit binds non-covalently to a pore formed by the pore-forming unit upon activation and oligomerization of the pore-forming unit, wherein the payload unit has at least 90% sequence identity to the amino acid sequence of SEQ ID NO: 2; wherein the pore-forming unit is a polypeptide derived from a first AB type of non-neurotoxic binary toxin, and wherein the target cell binding unit is a polypeptide derived from a second AB type of toxin, said second AB type being different from the first AB type, wherein the target cell binding unit binds to a neural cell; wherein the target cell binding unit and the pore-forming unit are covalently linked through the rigid synthetic linker to form a polypeptide.

In some embodiments, the pore-forming unit is a polypeptide or a polypeptide oligomer derived from the heavy chain of the pore-forming unit of Clostridium botulinum toxin C2. In some embodiments, the pore-forming unit comprises a native or modified heavy chain binding domain derived from a toxin other than C. botulinum toxin C2. In some embodiments, the polypeptide comprising target cell binding unit, rigid synthetic linker, and pore-forming unit has at least 90% sequence identity to the amino acid sequence of SEQ ID NO: 1. In some embodiments, the payload unit is not covalently bound to the target cell binding unit or the pore-forming unit. In some embodiments, the composition is administered to the subject by subcutaneous injection, transdermal (microneedling patch), intradermal, intramuscular, intravenous, intraperitoneal, or intrathecal administration. In some embodiments, the composition is administered in combination with one or more antiepileptic agents, tricyclic antidepressants, beta-blockers, or migraine headache prophylactic agents, the migraine headache prophylactic agents selected from propranolol, timolol, divalproex, valproic acid, topiramate, amitriptyline, nicotinamide adenine dinucelotide (NAD+), anti-CGRP or anti-CGRP receptor antibodies, or the combination thereof. In some embodiments, the composition is adapted to deliver molecules into peripheral sensory neurons, and wherein the composition does not substantially affect motor neurons. In some embodiments, the composition interacts with receptor ganglioside GT1b.

In some embodiments, the invention relates to a method for treating, preventing, or reducing the occurrence of headache disorders in a subject in need thereof comprising administering to the subject an effective amount of the composition of claim 1.

In some such embodiments, the patient has or is diagnosed with a headache disorder selected from episodic migraine headaches, tension-type headaches, cluster headaches, hemiplegic migraine headaches, or retinal migraine headaches. In some embodiments, the patient has or is diagnosed with chronic migraine. In some embodiments, the patient has at least four, but fewer than 15 migraine headache days per month. In some embodiments, the patient has failed or is intolerant to other classes of migraine headache prophylactic agents.

In some embodiments, the invention relates to a method for prophylactically treating headache disorders in a subject in need thereof comprising administering to the subject an effective amount of the composition of claim 1.

In some such embodiments, the patient has or is diagnosed with a headache disorder selected from chronic migraines, episodic migraine headaches, tension-type headaches, cluster headaches, hemiplegic migraine headaches, or retinal migraine headaches. In some embodiments, the pharmaceutical composition is administered in combination with one or more migraine headache prophylactic agents selected from propranolol, timolol, divalproex, valproic acid, topiramate, amitriptyline, nicotinamide adenine dinucleotide (NAD+), anti-CGRP or anti-CGRP receptor antibodies, or the combination thereof. In some embodiments, the pharmaceutical composition is administered in combination with an antiepileptic, a tricyclic antidepressant, and a beta-blocker. In some embodiments, the patient has failed or is intolerant to other classes of migraine headache prophylactic agents.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIGS. 1A-1C show NL-17 Mode of Action. FIG. 1A is a model of NL-17 domains. FIG. 1B is a bar graph showing the uptake of fluorescently labeled full length NL-17 or isolated domains by neuronal cell lines (N2A) was measured by cell sorting. Pre-treatment with GT1b promoted significant uptake relative to untreated controls. FIG. 1C shows the mode of action. The C2II domain binds neuron target cells via the C1 binding domain and translocates the payload C2I into the cytoplasm. C2II binds the cell surface via interactions with asparagine-linked glycans on the cell membrane. The C2I/C2II complex is internalized by clathrin and Rho-dependent mechanisms within endosomes. Acidification of the endosome causes membrane pore formation by C2II oligomers, and C2I dissociates and is transported into the cytoplasm, where it acts on G actin.

FIG. 2A shows protein domains of C. botulinum C1, C. botulinum C2II, fusion C2II-C1, and C2I. Numbers correspond to amino acid residues of each protein. (FIG. 2A) BoNT C1 has a linked enzymatic payload domain (light chain) and a binding/translocation domain (heavy chain). (FIG. 2B) The C2II binding/translocation component has four domains. Amino acid residue 182 indicates the trypsin cleavage position for activation of C2II into C2IIa. Domain 4 (D4) was removed to produce C2IIΔD4 as the translocation domain for C2II-C1. (FIG. 2C) The fusion C2II-C1 was made by linking C2IIΔD4 and BoNT C1 H_{CC} with an (EP)₁₀ linker flanked by glycine-serine residue pairs on both sides. Amino acid 182 is the activation site for C2II-C1. (FIG. 2D) The native C2I enzymatic payload of the C2 toxin and truncated C2It domain. Amino acids 299, 348, 387, and 389 are essential for ADP-ribosylation activity of C2I and are therefore not present in C2It.

FIG. 3 shows the experimental design to assay migraine-like behavior in mice.

FIGS. 4A-4B show that NL-17 prevents migraine-like behavior. C57BL/6 Female mice (8-12 wk) were pretreated (SC) with NL-17 or vehicle control (1×PBS) 2 hr before CGRP challenge. At time 0, animals were injected (IP) with vehicle (n=6) or CGRP [1 mg/kg](n=6). CGRP was reinjected after 23.5 and 47.5 hr to re-induce migraine-like behavior. Animals were evaluated using the light aversion and motility assay after 10 min, and 2, 4, 24, and 48 hr, and the grimace scale assay at 48 hr. The bar graph (FIG. 4A) depicts the average total light chamber time occupancy over the assay period. The bar graph (FIG. 4B) depicts the degree of grimace at 0.1, 2, 5, 24, and 48 hours after NL-17 administration. Error bars represent the standard deviation between replicates.

FIG. 5 shows that NL-17 treats acute/reactive migraine-like behavior. C57BL/6 Female mice (8-12 wk) were divided into 3 groups: Vehicle (n=3), CGRP [1 mg/kg](n=3), and NL-17 [30 ug/dose at 3 doses](n=3). CGRP was administered 30 min before Vehicle and NL-17, and behavior was monitored 4 hr later. Behavior was video recorded and scored blinded in 5-minute bins over 30-minute increments. Animals were evaluated using the light aversion and motility assay. Bars depict the average total light chamber time occupancy. Error bars represent the standard deviation between replicates.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

Unless otherwise specified, the experimental methods, detection methods, and preparation methods disclosed in the present invention all adopt the conventional molecular biology, biochemistry, microbiology, cell biology, genomics, and recombinant polynucleotides, chromatin structure and analysis, analytical chemistry, cell culture, recombinant DNA technology and related fields in the technical field. These techniques have been well described in the existing literature. For details, please refer to inter alia Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, Chromatin (P M Wassarman and A P Wolffe, eds.), Academic Press, San Diego, 1999; and METHODS IN MOLECULAR BIOLOGY, Vol. 119, Chromatin Protocols (PB Becker, ed.) Humana Press, Totowa, 1999, et al.; Cellular and Molecular Immunology, Ninth Edition, A. K. Abbas., et al., Elsevier (2017), ISBN 978-0323479783; Cancer Immunotherapy Principles and Practice, First Edition, L. H. Butterfield, et al., Demos Medical (2017), ISBN 978-1620700976; Janeway's Immunobiology, Ninth Edition, Kenneth Murphy, Garland Science (2016), ISBN 978-0815345053; Clinical Immunology and Serology: A Laboratory Perspective, Fourth Edition, C. Dorresteyn Stevens, et al., F. A. Davis Company (2016), ISBN 978-0803644663; Antibodies: A Laboratory Manual, Second edition, E. A. Greenfield, Cold Spring Harbor Laboratory Press (2014), ISBN 978-1-936113-81-1; Culture of Animal Cells: A Manual of Basic Technique and Specialized Applications, Seventh Edition, R. I. Freshney, Wiley-Blackwell (2016), ISBN 978-1118873656; Transgenic Animal Technology, Third Edition: A Laboratory Handbook, C. A. Pinkert, Elsevier (2014), ISBN 978-0124104907; The Laboratory Mouse, Second Edition, H. Hedrich, Academic Press (2012), ISBN 978-0123820082; Manipulating the Mouse Embryo: A Laboratory Manual, Fourth Edition, R. Behringer, et al., Cold Spring Harbor Laboratory Press (2013), ISBN 978-1936113019; PCR 2: A Practical Approach, M. J. McPherson, et al., IRL Press (1995), ISBN 978-0199634248; Methods in Molecular Biology (Series), J. M. Walker, ISSN 1064-3745, Humana Press; RNA: A Laboratory Manual, D. C. Rio, et al., Cold Spring Harbor Laboratory Press (2010), ISBN 978-0879698911; Methods in Enzymology (Series), Academic Press; Molecular Cloning: A Laboratory Manual (Fourth Edition), M. R. Green, et al., Cold Spring Harbor Laboratory Press (2012), ISBN 978-1605500560; Bioconjugate Techniques, Third Edition, G. T. Hermanson, Academic Press (2013), ISBN 978-0123822390; Methods in Plant Biochemistry and Molecular Biology, W. V. Dashek, CRC Press (1997), ISBN 978-0849394805; Plant Cell Culture Protocols (Methods in Molecular Biology), V. M. Loyola-Vargas, et al., Humana Press (2012), ISBN 978-1617798177; Plant Transformation Technologies, C. N. Stewart, et al., Wiley-Blackwell (2011), ISBN 978-0813821955; Recombinant Proteins from Plants (Methods in Biotechnology), C. Cunningham, et al., Humana Press (2010), ISBN 978-1617370212; Plant Genomics: Methods and Protocols (Methods in Molecular Biology), W. Busch, Humana Press (2017), ISBN 978-1493970018; Plant Biotechnology: Methods in Tissue Culture and Gene Transfer, R. Keshavachandran, et al., Orient Blackswan (2008), ISBN 978-8173716164.

In describing the exemplary embodiments, specific terminology will be resorted to for the sake of clarity. As used in the specification and the appended claims, the singular forms “a,” “an,” and “the” include plural references unless the context clearly dictates otherwise. For example, reference to a component is intended also to include the composition of a plurality of components. References to a composition containing “a” constituent are intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from one particular value/to another particular value.

As used herein, the term “antibody” is intended to denote an immunoglobulin molecule that possesses a “variable region” antigen recognition site. The term “variable region” is intended to distinguish such a domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). “Framework Region” or “FR” residues are those variable domain residues other than the hypervariable region residues as herein defined. The term antibody includes monoclonal antibodies (mAb), multi-specific antibodies, human antibodies, variant antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies). In particular, such antibodies include immunoglobulin molecules of any type (e.g., IgG, IgE, IgM, IgD, IgA, and IgY), class (e.g., IgG1, IgG2, IgG3, IgG4, IgA1, and IgA2), or subclass.

As used herein, the term “antigen binding fragment” of an antibody refers to one or more portions of an antibody that contain the antibody's Complementarity Determining Regions (“CDRs”) and optionally the framework residues that include the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins including the antibody's “variable region” antigen recognition site and a heterologous protein (e.g., a toxin, an antigen recognition site for a different antigen, an enzyme, a receptor or receptor ligand, etc.).

As used herein, the term “fragment” refers to a peptide or polypeptide including an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

As used herein, the term “modulate” relates to a capacity to alter an effect, result, or activity (e.g., signal transduction). Such modulation can be agonistic or antagonistic. Antagonistic modulation can be partial (i.e., attenuating, but not abolishing) or it can completely abolish such activity (e.g., neutralizing). Modulation can include internalization of a receptor following binding of a ligand or a reduction in expression of a receptor on the target cell. Agonistic modulation can enhance or otherwise increase or enhance an activity (e.g., signal transduction). In a still further embodiment, such modulation can alter the nature of the interaction between a ligand and its cognate receptor so as to alter the nature of the elicited signal transduction. For example, the molecules can, by binding to the ligand or receptor, alter the ability of such molecules to bind to other ligands or receptors and thereby alter their overall activity.

The term “substantially,” as used in the context of binding or exhibited effect, is intended to denote that the observed effect is physiologically or therapeutically relevant. Thus, for example, a molecule is able to substantially block an activity of a ligand or receptor if the extent of blockage is physiologically or therapeutically relevant (for example if such extent is greater than 60% complete, greater than 70% complete, greater than 75% complete, greater than 80% complete, greater than 85% complete, greater than 90% complete, greater than 95% complete, or greater than 97% complete). Similarly, a molecule is said to have substantially the same specificity and/or characteristic as another molecule, if such specificities and characteristics are greater than 60% identical, greater than 70% identical, greater than 75% identical, greater than 80% identical, greater than 85% identical, greater than 90% identical, greater than 95% identical, or greater than 97% identical).

As used herein, the “activating” or “stimulatory” signals encompass signals that result in enhancing an activity or enhancing signal transduction.

As used herein, “suppressive” signals refer to signals that suppress biological activity.

The term “endogenous concentration” refers to the level at which a molecule is natively expressed (i.e., in the absence of expression vectors or recombinant promoters) by a cell (which cell can be a normal cell, a cancer cell or an infected cell).

As used herein, the terms “treat,” “treating,” “treatment,” and “therapeutic use” refer to the elimination, reduction, or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, the term “prophylactic agent” refers to an agent that can be used disease, or to prevent a disorder or disease before any symptoms of such disorder or disease are detected. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the terms “individual,” “host,” “subject,” and “patient” are used interchangeably herein, and refer to a mammal, including, but not limited to, humans, rodents, such as mice and rats, and other laboratory animals.

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as a human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “variant” refers to a polypeptide or polynucleotide that differs from a reference polypeptide or polynucleotide, but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring, such as an allelic variant, or it may be a variant that is not naturally occurring.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within +2 is preferred, those within +1 are particularly preferred, and those within +0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that one amino acid can be substituted for another having a similar hydrophilicity value and still obtain a biologically equivalent, and in particular, an immunologically equivalent polypeptide. In such changes, the substitution of amino acids whose hydrophilicity values are within +2 is preferred, those within +1 are particularly preferred, and those within +0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Trp: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2, or Megalign (DNASTAR) software. Appropriate parameters for measuring alignment, including any algorithms needed to achieve maximal alignment over the full length of the sequences being compared, can be determined by known methods.

For purposes herein, the % sequence identity of a given nucleotide or amino acid sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “pharmaceutically acceptable carrier” encompasses any of the standard pharmaceutical carriers, such as a phosphate-buffered saline solution, water, and emulsions such as an oil/water or water/oil emulsion, and various types of wetting agents.

### II. Clostridium botulinum Compositions

Clostridium botulinum, a spore-forming, heat-resistant, anaerobic bacterium, produces a protein-based toxin (botulinum toxin) having several serotypes, known as A through G serotypes, of which serotype C has 3 subtypes, known as serotypes C1, C2, and C3. The C1 neurotoxin paralyzes people and animals in low doses by blocking acetylcholine release by neurons. Recovery is slow; treatment may require ventilation for multiple weeks before a person is able to breathe again. The C2 toxin is not neuro-active, and in avian intestinal tissue causes necrosis and hemorrhaging (Kurazono H, et al. Res Vet Sci. 1987 May; 42(3):349-53 (1987)). The C3 toxin is the least characterized of the C sub-serotypes.

Most toxin-based delivery systems are multi-domain proteins that bind target cells and translocate material (payloads) across the lipid bilayer into the cytosol of the targeted cell. These systems are altered AB-type toxins, consisting of a payload domain (A) and a binding/translocation domain (B). The A and B domains can be covalently linked by a polypeptide or disulfide bond that is later cleaved during the translocation step. Non-covalently linked (binary) A and B toxin domains are transcribed and translated independently and associate prior to exerting toxicity. The Clostridium botulinum C2 toxin (C2) is not a neurotoxin, but it has a binary AB toxin design.

Compositions and methods for delivering molecular payloads to the cytosol of target cells are disclosed. Bacteria have evolved mechanisms to target cells and deliver toxic payloads to the cytosol of target cells. This mechanism may be modified and engineered to deliver beneficial payloads. Payloads can be small molecules, protein toxins, biologically active peptides, enzymes, or even radionuclides.

In general, there are two classes of AB-type bacterial toxins: linked and unlinked (binary). Linked toxins typically have a single chain protein containing both a toxin domain and a binding/translocation domain. Binary toxins typically have two separately expressed protein molecules, where the binding/translocation domain and the toxin domain assemble via non-covalent interactions.

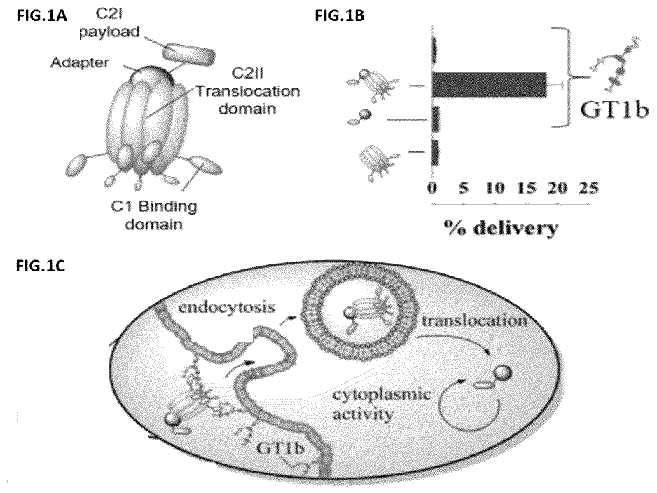

The native C2 toxin is composed of two separate proteins. The B domain protein (C2II) binds target cells and translocates the A domain (C2I, the payload). The A domain is an ADP-ribosyltransferase that causes cell rounding and apoptosis, initiated by the ADP-ribosylation of cytoplasmic actin (FIG. 1A). C2II monomers are proteolytically processed to remove a 20 kDa segment from the N-terminus, which activates the binding/translocation domain into C2IIa. C2IIa monomers then spontaneously oligomerize and bind the cell surface via interactions with asparagine-linked glycans on the cell membrane. The A domain, C2I, binds to the C2IIa oligomers and the C2IIa/C2I complex is internalized by clathrin and Rho-dependent mechanisms. Acidification of the early endosome triggers the formation of membrane pores by C2IIa oligomers, allowing C2I to be transported into the cytoplasm.

### III. NL-17 Composition

Provided herein are compositions for treating headaches. The invention provides methods of making NL-17, resulting in higher yields at lower cost.

NL-17 is comprised of two protein subunits, C2II-C1 and C2I (FIG. 1A). NL-17 has similarity to Botox through its shared origin from Clostridia botulinum, though from different serotypes than Botox. Botox is from serotypes A and B. NL-17 is from serotype C. Unlike Botox, NL-17 is an engineered protein, not a natural protein; the C-terminal region of C2II has a recombinant region from C1 that replaces the native C2II C-terminal region. The C1 region interacts tightly with a receptor ganglioside called GT1b, present in high amounts on sensory (not motor) neurons. Upon contact with GT1b, NL-17 undergoes receptor-mediated endocytosis, entering a membrane vesicle within the sensory neuron. The C2II-C1 component of NL-17, which is present as a heptameric oligomer, fuses with the vesicle membrane, forming a pore through which the C2I component of NL-17 diffuses to enter the sensory neuron cytoplasm. The cytoplasm contains actin present in two forms, monomeric G-actin and polymeric F-actin. C2I, which is an ADP ribosyltransferase, ribosylates G-actin, shifting the treadmilling balance between G and F-actin, thereby depleting F-actin. Because ion channels are in direct contact with F-actin, the depletion of F-actin causes a transient reduction in Calcium ion uptake. Since Calcium ion uptake (influx) is required for sensory neuron activation and thereby pain signaling, signaling is reduced and neurons become quiescent, ceasing pain signaling.

For therapeutic development, engineering of a binary toxin has certain advantages because the binding/translocation domain and the payload domain may be separately expressed and purified. The C2 toxin from C. botulinum is a binary structure, but is nonspecific as it binds a variety of cells and necessitates N-linked glycans for intoxication (i.e., it is not a specific neurotoxin). Disclosed here are methods to engineer the C2 toxin binding domain by retargeting to neural cells to produce NL-17. More specifically, the target binding domain from the C1 botulinum neurotoxin may be used. The binding domain from the C1 botulinum neurotoxin has been previously applied as a targeting component for drug delivery to peripheral neural tissue in linked toxin designs and as liposomal surface modifications.

In one embodiment, binding domain replacement of the C2 toxin requires that the retargeted binding/translocation component retain its ability to oligomerize upon activation, bind to the new targeting moiety on the cell surface, and translocate the payload into the cytosol of the target cell. The natural binding domain of the C2 toxin is located at the C-terminal end of the molecule and is designated as D4 (U.S. Ser. No. 10/633,643B2). In one aspect, D4 is not required for oligomerization because translocation pores can be formed in artificial membranes even when D4 is absent. In another aspect, D4 is deleted from C2II and replaced with the BoNT C1 binding domain that would target the molecule to peripheral neurons. The BoNT C1 Hcc (U.S. Ser. No. 10/633,643B2) preferentially binds gangliosides GT1b and GD1b.

In another embodiment, BoNT/A N-terminal heavy chain domain (HCN) is not included in the chimeric C2II-C1. It has been shown that HCN may assist in the orientation of the toxin for association with the membrane by interacting with phosphatidylinositol phosphates. It is shown that although HCN may be active in native BoNT translocation, it is not required in a chimeric C2II-C1 translocation event.

In another embodiment, the binding domain is taken from a linked toxin and inserted into the binding/translocation domain of a binary toxin. This configuration retargets the resulting molecule to neurons while maintaining the C2 toxin's mechanism of activation and translocation. It should be noted that a similarity exists between BoNT and C2 endocytosis and translocation mechanisms in that a clathrin/rho/dynamin-mediated endocytic-endosomal entry pathway characterized by pH-dependent protein conformational changes is implicated for both toxins.

In another embodiment, attempts have been made to express a soluble C2II-C1 fusion protein that oligomerizes upon activation with trypsin. Direct fusion of the C1 H_{CC} domain was not successful due to solubility problems. To remedy this limitation a flexible glycine-serine linker (G₄S)ₙ was used but encountered similar issues. Finally, use of a rigid (EP)₁₀ linker resulted in a soluble fusion protein that is compatible with activation and oligomerization. SDS-PAGE confirmed that the C2II-C1 fusion protein could be activated by limited trypsin digestion and then oligomerize. Western blotting is used to confirm that the C1 H_{CC} domain is incorporated into the oligomeric species. BoNT C1 antigenicity specific to the C2II-C1 oligomer and a decrease in electrophoretic mobility in comparison to C2IIΔD4 demonstrate that C1 H_{CC} at the C-terminus of C2II-C1 does not prevent oligomerization and is compatible with limited trypsin digestion.

To quantify and visualize binding and internalization of a payload by C2II-C1, a fluorescently labeled C-terminally truncated C2I-based payload, C2It (U.S. Ser. No. 10/633,643B2), was constructed for use in flow cytometry and microscopy experiments. C2It, which was composed of amino acids 1-226 of C2I (not containing the ADP-ribosylating active site residues), was fluorescently labeled in two separate versions with Alexa Fluor 488 (C2It-488) and 568 (C2It-568) by amine reactive chemistry. Previously, BoNT C1 Hc entry was shown to be GT1b-dependent in N2A cells that were artificially enriched for GT1b³⁰, and this strategy was adapted to study targeting by the C2II-C1 fusion protein. If the engineered B component, C2II-C1, were activated, oligomerized and associated with the fluorescently labeled A component, C2It, GT1b-dependent uptake of fluorescently labeled C2It should be observed. This cellular model does not employ electrostimulation as previously described to enhance BoNT C1 intoxication because entry alone was presumed to be sufficient for the non-neural-specific C2 component of the fusion to promote translocation activity. For flow cytometry, a culture of N2A cells was enriched with GT1b while another was not; both cultures were incubated with activated C2II-C1 and C2It-488, and then both cultures were treated with pronase to remove extracellular proteins prior to being analyzed. Cells with intracellular fluorescence above 10³ absorbance units were counted by flow cytometry, and repeated results showed that N2A cell populations enriched with the binding domain receptor GT1b preferentially took up C2II-C1-delivered fluorescent C2It (U.S. Ser. No. 10/633,643B2). The results shown in U.S. Ser. No. 10/633,643B2 indicate that the BoNT C1 H_{CC} can be used to replace another toxin-binding domain and result in a GT1b-dependent entry specificity. C2It-568 preferentially enters GT1b-enriched cells and does not colocalize with fluorescently labeled early endosomes. Escape from the early endosome by transport of C2It through the pore created by the translocation domain is a determinant of payload delivery to the cytosol. These results are consistent with the expected association between the engineered payload and binding/translocation domain by GT1b-specific delivery of C2It by C2II-C1. Lack of colocalization of early endosomes with C2It-568 (U.S. Ser. No. 10/633,643B2) provides evidence to pursue other payloads with the intent of cytosolic delivery to manipulate the cytosome.

To deliver an active enzyme to the cytosol by the C2II-C1 fusion, the native C2 toxin A component, C2I, may be produced. The C2I enzyme is known to cause cell rounding in eukaryotic cells by ADP-ribosylation of cytosolic actin. The effect of C2I is tested after delivery by C2II-C1 to human glioblastoma A172 and HeLa cell lines that are enriched with the ganglioside GT1b. A greater than two-fold increase in cell rounding of GT1b-enriched cell populations is found for both cell lines when compared to controls lacking GT1b enrichment. By comparison, payload-induced cell rounding of synchronized HeLa cells in the presence of the fusion translocator C2II-C1 is less efficient than reported by Barth et al. (Barth, H. et al. J. Biol. Chem. 275, 18704-18711 (2000)) in the presence of the native C2II translocation domain. A truncated form of C2II-C1 characterized during expression may have been incorporated into C2II-C1 oligomers, which may result in a decrease in binding efficiency. Although an apparent lack of monomeric C2II-C1 in final purification fractions is evident by SDS-PAGE, it is possible that monomeric C2II-C1 dissociated or was not incorporated into oligomers, and competes for binding with the functional form of the oligomeric delivery system. These findings confirm the native cytosolic activity of the C2I enzyme specifically delivered by C2II-C1 in a GT1b-dependent manner.

In another embodiment, alternate payloads based on modified C2. It may be used in delivery applications of the C2II-C1 fusion protein affecting the natural targets of BoNTs (U.S. Ser. No. 10/633,643B2). A minimal region of amino acid residues 1-87 in the C2I component is required for complementary activity with the native C2II translocation domain. Translocation of non-canonical polypeptides may also be possible with modified C2I, similar to payload development work recently conducted with anthrax lethal factor. This disclosure provides the basis for exploring other binding specificities and payload domains for additional applications in treating headaches.

### IV. NL-17 Sequences

The present disclosure provides chimeric toxin-based delivery compositions (or systems) for delivering a payload (or agent) to a target cell. In one embodiment, the composition may contain a target cell binding unit, a pore-forming unit, and a payload unit, with or without other additional components. Examples of the chimeric toxin-based delivery compositions (or systems) described herein are described in U.S. Ser. No. 10/633,643B2 and U.S. Ser. No. 11/118,170B2.

In one embodiment, the pore-forming unit may be the same as the pore-forming unit of known toxins, for example, toxins from Clostridium botulinum. In another embodiment, the pore-forming unit may be derived from the pore-forming unit of known toxins with modifications. In another embodiment, the pore-forming unit may be any protein that may function as a pore-forming unit.

The payload unit may contain the agent to be delivered to the target cell. In one aspect, the payload unit may bind non-covalently to the pore-forming unit, or to the linked pore-forming unit and target cell-binding unit. In another aspect, the pore-forming unit and target cell-binding unit are linked covalently.

In one embodiment, the payload unit may be the same as the payload unit of known toxins, for example, toxins from Clostridium botulinum. In another embodiment, the payload unit may be derived from the payload unit of known toxins with modifications. In another embodiment, the payload unit may be any protein that may function as a payload unit for the delivery of the agent.

In another embodiment, the target cell binding unit may contain a specific target cell binding ligand selected from the group consisting of antibody, antibody fragment, affibody, growth factor, a receptor-binding ligand, or combinations thereof. In another embodiment, the target cell binding unit may contain a native or modified binding domain derived from a heavy chain of C. botulinum toxin other than C2.

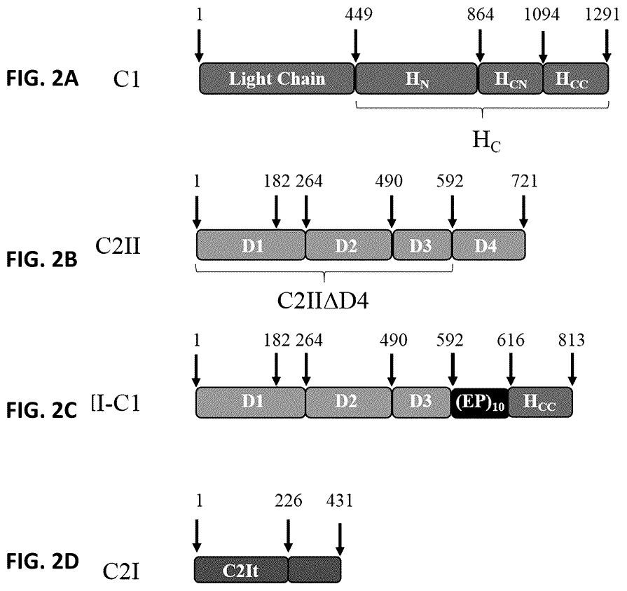

In another embodiment, the target cell binding unit preferentially binds to a neural cell. In another embodiment, the composition preferentially delivers the agent to neural cells. In another embodiment, the target cell binding unit is the binding domain of the heavy chain of C. botulinum neurotoxin C1 (C1 Hcc) (See FIG. 2C).

In another embodiment, the pore-forming unit may be a polypeptide derived from a first type of non-neurotoxic (i.e., not specifically targeting neuron) toxin, and the target cell binding unit may be a polypeptide derived from a second type of toxin, wherein the second type is different from the first type of toxin. In another embodiment, the first type of non-neurotoxic toxin may be a binary toxin.

In another embodiment, the pore-forming unit may be a polypeptide derived from a first Clostridium toxin sub-serotype, while the target cell binding unit is a polypeptide derived from a second Clostridium toxin sub-serotype, wherein the second sub-serotype is different from the first sub-serotype. In another embodiment, the pore-forming unit is a polypeptide derived from the pore-forming unit of Clostridium botulinum toxin C2 (See FIG. 2C).

In another embodiment, the pore-forming unit may contain a native or modified domain derived from a toxin other than C. botulinum toxin C2. In one aspect, the pore-forming unit may contain a native or modified pore-forming domain derived from a toxin selected from the group consisting of Clostridium perfringens alpha-, beta-, epsilon-, and iota-toxin, Clostridium spiroforme Iota-like toxin, anthrax toxin, and combinations thereof.

In one embodiment, the pore-forming unit is a polypeptide derived from the pore-forming unit of Clostridium botulinum toxin C2, while the target cell binding unit may contain a native or modified binding domain derived from a toxin other than C. botulinum toxin C2. In one aspect, the target cell binding unit may contain a native or modified binding domain derived from a toxin selected from the group consisting of C. botulinum neurotoxins, Clostridium perfringens toxins alpha, beta, epsilon and iota toxin, Clostridium spiroforme Iota-like toxin, cholera toxin, anthrax toxin, shiga toxin, shiga-like toxin, diphtheria toxin, ricin, exotoxin A, and combinations thereof.

In another embodiment, the payload unit is not covalently bound to the target cell binding unit or the pore-forming unit. In another embodiment, the payload unit is a polypeptide derived from Clostridium botulinum toxin C2 (See FIG. 2D, SEQ ID NO: 2).

In another embodiment, the agent comprises at least one member selected from the group consisting of a therapeutic agent, a diagnostic agent, an imaging agent, and combinations thereof. In another aspect, the agent may contain at least one member selected from the group consisting of a toxin, a cell cycle blocker, an apoptosis inducing agent, an inhibitor of DNA replication, an inhibitor of RNA synthesis, an inhibitor of protein synthesis, an enzyme, a protein binding agent, an antibody, a neutralizing antibody, a labeling agent, magnetic beads, and combinations thereof.

In another embodiment, the agent comprises an ADP-ribosyltransferase. In another embodiment, the agent comprises C2I from Clostridium botulinum toxin C-2. In another embodiment, the agent comprises a fluorescent agent for labeling or monitoring the target cell.

In one embodiment, the target cell may be a cancer cell. In another embodiment, the target cell may be a neuron. In another embodiment, the target cell may be a cell of a brain tumor, a cell of a neuroblastoma, a cell of a retinoblastoma, a peripheral neuron, a motor neuron, a sensory neuron, or combinations thereof.

In another embodiment, the engineered payload-delivery composition may include a target-cell binding unit that is covalently bound to a pore-forming unit, and a payload portion adapted with a region capable of non-covalently binding to the pore-forming unit. In another embodiment, a polypeptide (SEQ ID NO: 1) is disclosed, which may contain the target-cell binding unit covalently bound to the pore-forming unit linked by a linker (EP)₁₀, wherein the target-cell binding unit is the binding domain of the heavy chain of C. botulinum neurotoxin C1 (C1 Hcc) and the pore-forming unit is a polypeptide derived from the pore-forming unit of Clostridium botulinum toxin C2.

In another embodiment, the active payload region is bound to the pore-forming unit through a coupling region derived from the light-chain payload portion of botulinum toxin C-2. In another aspect, the target-cell binding unit is derived from the target-cell binding unit of botulinum toxin C-1.

In another embodiment, the target cell binding unit is the binding domain from the heavy chain of C. botulinum neurotoxin C1 (C1 Hcc), while the pore-forming unit is the pore-forming unit of Clostridium botulinum toxin C-2, and the payload unit comprises C2I from Clostridium botulinum toxin C-2.

In another embodiment, in a composition for delivering an agent to a target cell comprising a payload unit and a binding unit comprising a target-cell binding unit covalently bound to a pore-forming unit linked by a linker, the binding unit comprises a polypeptide having at least 80, 90, 95, 99%, or 100% sequence identity to SEQ ID NO: 1, and the payload unit comprises a polypeptide having at least 80, 90, 95, 99%, or 100% sequence identity to amino acid sequence of SEQ ID NO: 2.

In another embodiment, the disclosed composition may be administered to a subject by injection, wherein the subject contains the target cell(s).

In another embodiment, a polynucleotide encoding a polypeptide is disclosed, wherein the polypeptide has at least 80%, 90%, 95%, 99%, or 100% identity to a polypeptide selected from the group consisting of SEQ ID NOs 1-2. In one aspect, the polynucleotide may be carried on a vector. In one aspect, the vector may be capable of replicating itself.

In another embodiment, a host cell comprising the polynucleotide is also disclosed. The host cell may be used to produce the composition for delivering the agent to a target cell. In another embodiment, the host cell may be introduced into a subject for delivery of the agent. In another embodiment, for purpose of this disclosure, the host cell may be a bacterium, or a virus.

A list of sequences of SEQ ID NOs: 1-2 is shown below.

(SEQ ID NO: 1)
MANANRDTDRDGIPDEWEINGYTVMNQKAVAWDDKFAANGYK
KYVSNPFKPCTANDPYTDFEKVSGQIDPSVSMVARDPMISAY
PIVGVQMERLVVSKSETITGDSTKSMSKSTSHSSTNINTVGA
EVSGSLQLAGGIFPVFSMSASANYSHTWQNTSTVDDTTGESF
SQGLSINTAESAYINPNIRYYNTGTAPVYNVTPTTTIVIDKQ
SVATIKGQESLIGDYLNPGGTYPIIGEPPMALNTMDQFSSRL
IPINYNQLKSIDNGGTVMLSTSQFTGNFAKYNSNGNLVTDGN
NWGPYLGTIKSTTASLTLSLPDQTTQVAVVAPNFSDPEDKTP
RLTLEQALVKAFRLEKKNGKFYFHGMEISANQKIQVFLDRNT
NVDFENQLKNTANKDIMNCIIKRNMNILVKVITGSEPEPEPE
PEPEPEPEPEPEPGNNINDSKILSLQNRKNTLVDTSGYNAEV
SEEGDVQLNPIFPFDFKLGSSGEDRGKVIVTQNENIVYNSMY
ESFSISFWIRINKWVSNLPGYTIIDSVKNNSGWSIGIISNFL
VFTLKQNEDSEQSINFSYDISNNAPGYNKWFFVTVTNNMMGN
MKIYINGKLIDTIKVKELTGINFSKTITFEINKIPDTGLITS
DSDNINMWIRDFYIFAKELDGKDINILFNSLQYSTNVVKDYW
GNDLRYNKEYYMVNIDYLNRYMYANSRQIVFNTRRNNNDFNE
GYKIIIKRIRGNTNDTRVRGGDILYFDMTINNKAYNLFMKNE
TMYADNHSTEDIYAIGLREQTKDINDNIIFQIQPMNNTYYYA
SQIFKSNFNGENISGICSIGTYRFRLGGDWYRHNYLVPTVKQ
GNYASLLESTSTHWGFVPVSE
(SEQ ID NO: 2)
MAIIKEPIDFINKPESEAKKWGKEEEKRWFTKLNNLEEVAVN
QLKNKEYKTKIDNFSTDILFSSLTAIEIMKEDENQNLFDVER
IREALLKNTLDRDAIGYVNFTPKELGINFSIRDVELDRDISD
ETLDKVRQQIINQEYTKFSFISLGLNDNSINESVPVIVKTRV
PTTFDYGVLNDKETVSLLLNQGFSIIPESAIITTIKGKDYIL
IEGSLSQELDFYNKGSEAWGAENYGDYISKLSHEQLGALEGY
LHSDYKAINSYLRNNRVPNNDELNKKIELISSALSVKPIPQT
LIAYRRVDGIPFDLPSDFSFDKKENGEIIADKQKLNEFIDKW
TGKEIENLSFSSTSLKSTPLSFSKSRFIFRLRLSEGTIGAFI
YGFSGFQDEQEILLNKNSTFKIFRITPITSIINRVTKMTQVV
IDAEVIQNKEI

### V. Methods of Treating Acute Pain

Provided herein are methods of treating headaches comprising administering NL-17 to a subject in need thereof. Also provided are methods of preventing migraines by preemptively treating a subject prone to headaches and/or migraines with NL-17.

With an estimated 30% of the population worldwide affected, pain is one of the most prevalent health problems in the world. Pain affects 56% of American adults, more than diabetes, heart disease, and cancer combined. Acute pain due to injuries, accidents, labor, and childbirth, or surgeries, represents a significant concern for 67.5% of patients admitted to hospitals. There are nearly 50 million surgeries in the US per year that involve pain management.

Unrelieved acute pain is one of the risk factors in the development of chronic pain, with 10%-50% of patients developing persistent pain after different operations. Specifically, there are over a million orthopedic surgeries performed each year in the United States. Total hip and knee arthroplasties are common surgeries in orthopedics, with more than 300,000 hip replacements and 600,000 knee replacements performed each year.

While pain is sensed in the brain, the event that triggers the sensation occurs in the periphery, typically by transient or permanent damage to sensory neurons (also called nociceptors), leading to their activation. The idea for NL-17 arose from understanding how pain works and the need to avoid side effects such as addiction, suffocation, and intestinal damage caused by opioids. That promoted years of protein design and engineering efforts. Eventually, a “rationale” drug was created that had the envisioned properties. NL-17 was designed to be administered to peripheral sensory neurons located at the site where pain signaling originates. NL-17 targets only sensory neurons in the body's peripheral regions and reduces their excessive activity. Unlike opioids, which are consumed orally and act systemically, NL-17 is administered by injection or through the use of a patch. Because of its large size and mode of administration, it diffuses poorly and cannot cross the blood-brain barrier and therefore cannot lead to addiction.

NL-17 as a biologic, is multifunctional. It targets only sensory neurons, not motor neurons, and therefore has no effect on muscles. It becomes active only once it enters sensory neurons. Together, these two functions limit side effects and distinguish NL-17 from all other pain drugs. Once inside the sensory neuron, NL-17 modifies the internal actin-based skeleton to reduce neural signaling. Cell targeting combined with intracellular activity minimizes side effects and improves the likelihood of positive clinical trial outcomes.

NL-17 is locally administered at the site of pain, by injection, patch, or nerve block, not systemic, patch, or nerve block, no CNS effect. The drug acts like a dimmer switch on neurons without blocking sensation. Importantly, NL-17 is reversible.

NL-17 is a very large protein, not a small molecule like OxyContin or Ibuprofen (Advil). Its large size prevents it from diffusing inside the body and also blocks its entry into the brain. These features prevent addiction and make NL-17 unlike all competitor drugs for severe pain.

Sensory neuron targeting avoids side effects such as toxicity and immune reactions. All the competitor drugs of NL-17 are untargeted and have severe side effects that result from their interactions with cells and organs that have nothing to do with pain.

### VI. Methods of Treating Migraines and Headaches

Migraine is a complex, common neurological condition that is characterized by severe, episodic attacks of headache and associated features, which may include nausea, vomiting, sensitivity to light, sound or movement. In some patients, the headache is preceded or accompanied by sensory warning signs or symptoms (i.e. auras). The headache pain may be severe and may also be unilateral in certain patients. Migraine attacks are disruptive to daily life and cost billions of dollars each year in missed work days and impaired performance (Modi S, Lowder D M. Am. Fam. Physician, Vol. 73:72-78 (2006)).

Migraine is a highly prevalent disease worldwide with approximately 15% of the European population and 12% of the United States population suffering from migraine attacks (Lipton R B, et al. Neurology. 30; 68(5):343-9 (2007)). Additionally, migraines have been found to be associated with a number of psychiatric and medical comorbidities such as depression and vascular disorders (Buse D C, et al. J Neurol Neurosurg Psychiatry. 81(4):428-32 (2010); Bigal, M. E., et al. Neurology, 72, 1864-1871 (2009)).

Migraine headache is commonly treated acutely, primarily with analgesics and a class of drugs called triptans (Humphrey P P, et al. Ann N Y Acad Sci. 600:587-98; discussion 598-600(1990); Houston, D. S., Vanhoutte, P. M. Drugs 31, 149-163 (1986)). The triptans, which are selective serotonin 5-HT1B/1D agonists, are effective drugs for acute migraine and are generally well tolerated, but are contraindicated in the presence of cardiovascular disease due to their potential for coronary vasoconstriction. In addition, many migraine patients do not respond favorably to triptans. In a meta-analysis of 53 trials, up to a third of all people with migraine and 40% of all migraine attacks did not respond to triptans (Ferrari M D, et al. Lancet. 17; 358(9294):1668-75. (2001)).

Migraine prophylaxis is an area of large unmet medical need. Approximately 40% of the migraine patient population would benefit from preventive therapy (Lipton et al., 2007). However, only approximately 12% of patients receive any preventive therapy due in part to limited efficacy and significant tolerability and safety issues with available preventive therapies. Topiramate, an anticonvulsant that blocks voltage-dependent sodium channels and certain glutamate receptors (AMPA-kainate), is the medication most often used for migraine prophylaxis in the United States. Topiramate is the only migraine prophylactic agent with demonstrated efficacy in both episodic and chronic migraine patients through randomized placebo-controlled trials (Diener H C, et al. Cephalalgia. 2007 July; 27(7):814-23 (2007); Silberstein S D, et al. Headache. 47(2):170-80 (2007)). However, approximately 50% of patients fail to respond to topiramate and it is poorly tolerated. Common adverse events associated with topiramate treatment include paresthesia, anorexia, and cognitive adverse events, including psychomotor slowing, somnolence, language difficulties, and difficulties with memory and concentration (Brandes J L, et al. JAMA. 25; 291(8):965-73 (2004); Adelman J, et al. Pain medicine. 9(2):175-85(2008); Silberstein S D, et al. Arch Neurol. 261(4):490-5 (2004). In an open-label, flexible-dose study, 20% of patients withdrew from topiramate because of adverse effects (Nelles et al., Headache, Vol. 49:1454-1465, 2009).

### VII. Methods of Administration

In certain embodiments of the methods of the invention, the number of migraine attacks experienced by the patient is reduced following administration of NL-17 as compared to the number of migraine attacks experienced by the subject prior to treatment or the number of migraine attacks experienced by a control subject. As used herein, the term “migraine attack” refers to an episode of any migraine headache as defined herein. A migraine attack that is interrupted by sleep or temporarily remits and then recurs within 48 hours is generally considered to be a single attack. Similarly, a migraine attack that is successfully treated with acute migraine-specific medication but relapses within 48 hours is also considered to be a single attack. In some embodiments, the number of migraine attacks is reduced in the patient by at least about 25%, at least about 30%, at least about 40%, at least about 50%, at least about 60%, at least about 70%, or at least about 75% following administration of NL-17 as compared to the number of attacks prior to treatment or the number of attacks in a control subject.

In some embodiments, the therapeutic regimens of the invention ameliorate one or more symptoms associated with migraine in a patient in need thereof. For instance, administration of NL-17 to the patient according to the methods described herein reduces the occurrence of or treats one or more symptoms in the patients as compared to a control subject (i.e. a subject not receiving NL-17). Symptoms that can be ameliorated or treated with the methods of the invention include, but are not limited to, vasomotor symptoms (e.g. hot flashes, facial flushing, sweating, and night sweats), photophobia (sensitivity to light), phonophobia (sensitivity to sound), sensitivity to smells, vertigo, dizziness, nausea, vomiting, and headache pain.

In some aspects, the methods of the invention comprise administering to a patient a pharmaceutical composition comprising a therapeutically effective amount of NL-17. A “therapeutically effective amount” refers to an amount sufficient to remedy migraine headache or symptoms, particularly a state or symptoms associated with migraine headache, or otherwise prevent, hinder, retard, or reverse the progression of migraine headache or any other undesirable symptom associated with migraine headache in any way whatsoever. In certain embodiments, a therapeutically effective amount is an amount sufficient to prevent or delay the onset or recurrence of migraine headache or reduce the likelihood of its onset or recurrence.

Thus, in some embodiments, NL-17 is administered to the patient at a total dose of about 35 mg to about 210 mg per month. For instance, the dose of NL-17 can be about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or about 210 mg per month. Ranges between any and all of these endpoints are also contemplated, for example about 35 mg to about 70 mg, about 40 mg to about 90 mg, about 50 mg to about 80 mg, about 35 mg to about 140 mg, about 70 mg to about 140 mg, about 50 mg to about 100 mg, about 70 mg to about 210 mg, about 140 mg to about 210 mg, or about 150 mg to about 200 mg per month. In some such embodiments, the monthly dose of NL-17 is similar among patients regardless of body weight. In other words, in these embodiments, the monthly dosage of NL-17 is a total dose and is not adjusted for a patient's body weight. In one embodiment, NL-17 is administered to the patient at a total dose of about 70 mg to about 140 mg per month. In certain embodiments of the methods described herein, NL-17 is administered to the patient at a total dose of about 70 mg per month. In other embodiments, NL-17 is administered to the patient at a total dose of about 140 mg per month.

In certain embodiments, the monthly dose of NL-17 may be based upon a patient's body weight. For example, in some embodiments, the monthly dose of an NL-17 thereof may range from about 0.3 mg/kg to about 3.5 mg/kg of body weight, from about 0.5 mg/kg to about 3 mg/kg of body weight, or from about 1 mg/kg to about 2.5 mg/kg of body weight. For instance, the monthly dose of NL-17 may be about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.2 mg/kg, about 3.3 mg/kg, about 3.4 mg/kg, or about 3.5 mg/kg of body weight. In one embodiment, the monthly dose of NL-17 is about 0.8 mg/kg to about 1.2 mg/kg of body weight. In another embodiment, the monthly dose of NL-17 is about 1.6 mg/kg to about 2.2 mg/kg of weight.

The dose of NL-17 can be administered in a single administration or divided among multiple administrations over the course of the dosing frequency period. For example, in certain embodiments, the therapeutically effective dose of NL-17 is administered in a single administration each frequency period. Thus, in some embodiments, any of the doses of NL-17 described herein can be administered to the patient once a month (QM dosing). Patients on a QM dosing regimen are typically administered the NL-17 every 24 to 36 days, preferably every 28 to 35 days, more preferably, every 28 to 31 days, or even more preferably, every 28 days or every 30 days. In these and other embodiments, the monthly dose is administered to the patient as a bolus injection, for example, using a self-injection device as described herein. For instance, a monthly dose of 70 mg can be administered to the patient as a single bolus injection of 70 mg, optionally with an autoinjector, pen injector, or pre-filled syringe containing the 70 mg dose. In certain embodiments, the monthly dose is given in two or more consecutive injections. By way of example, a monthly dose of 70 mg can be administered to the patient in two consecutive injections of 35 mg, optionally with two injection devices (e.g., autoinjectors, pen injectors, or pre-filled syringes) containing a 35 mg dose. Similarly, a monthly dose of 140 mg can be administered to the patient in two consecutive injections of 70 mg, optionally with two injection devices (e.g., autoinjectors, pen injectors, or pre-filled syringes) containing a 70 mg dose. Consecutive injections given within the period of a single day are considered to be a single administration. In other words, by way of example, a single bolus injection of 70 mg and two consecutive injections of 35 mg within a one-day period would both be considered a single administration of a 70 mg dose.

In alternative embodiments, the doses of NL-17 are divided among two or more administrations over the course of the dosing frequency period. For example, for a dosing frequency period of one month, the monthly dose may be divided into four doses and administered on a weekly basis or divided into two doses and administered every two weeks. Any of the doses of NL-17 described herein can be divided among two or more administrations. The number of administrations and intervening interval can be adjusted for a particular patient depending on the type and severity of migraine (e.g. episodic or chronic), the age of the patient, the physical health of the patient, concomitant treatment with other medications, and/or the presence of other conditions.

In certain embodiments, the dosing frequency period for the doses of NL-17 that are described herein is monthly. In other words, the dosages of NL-17 are monthly dosages, but can be administered in a single administration (i.e., once a month; QM dosing) or divided among multiple administrations over the course of the month (e.g., 12 the monthly dose administered every two weeks). In some embodiments, the dosing frequency is once every 2 months (Q2M dosing). In other embodiments, the dosing frequency is once every 3 months (Q3M dosing).

In some embodiments of the methods of the invention, NL-17 is administered to the patient over the course of a set treatment period. A “treatment period” begins upon administration of the first dose of NL-17 and ends upon administration of the final dose of NL-17. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48, 60, 64, or more months. In one particular embodiment, the treatment period is at least about 6 months and produces a statistically significant reduction in the frequency, duration, or severity of migraine headache in the patient as compared to untreated subjects.

Administration of an NL-17 according to the methods of the invention preferably causes few or no adverse side effects in the patient. As used herein, the term “adverse side effect” refers to any abnormality, defect, mutation, lesion, degeneration, harmful or undesirable reaction, symptom, or injury, which may be caused by taking the drug. In some embodiments, administration of the NL-17 does not substantially cause one or more adverse side effects associated with other migraine prophylactic treatments (e.g. amitriptyline, divalproex, valproic acid, propranolol, timolol, topiramate, and botulinum toxin A). Side effects associated with other migraine prophylactic treatments include, but are not limited to, fatigue, nausea, dizziness, insomnia, depression, reduced exercise tolerance, tremor, paresthesia, teratogenicity, and cognitive difficulty. In other embodiments, administration of NL-17 is associated with a lower rate or number of adverse side effects as compared to the rate or number of adverse side effects associated with other migraine prophylactic treatments. In yet other embodiments, administration of NL-17 is associated with a lower rate of discontinuation due to adverse side effects as compared to the rate of discontinuation due to adverse side effects associated with other prophylactic treatments. In certain embodiments, the number and type of adverse side effects associated with administration of NL-17 are not statistically different than the number and type of adverse side effects associated with administration of a placebo. In some embodiments, administration of NL-17 is not associated with an adverse event higher than grade 2 as assessed by the Common Terminology Criteria for Adverse Events v4.0 (CTCAE). In other embodiments, administration of an NL-17 is not associated with an adverse event higher than grade 1 as assessed by the CTCAE.

### VIII. Routes of Administration

Pharmaceutical compositions containing the nontoxic engineered protein derived from a botulinum toxin complex can be administered by parenteral (intramuscular, intraperitoneal, intravenous (IV) or subcutaneous injection), transdermal (microneedling patch), intradermal, or intrathecal routes of administration or using bioerodible inserts and can be formulated in dosage forms appropriate for each route of administration.

In some in vivo approaches, the compositions disclosed herein are administered to a subject in a therapeutically effective amount. The precise dosage will vary according to a variety of factors such as subject-dependent variables (e.g., age, immune system health, etc.), the disease, and the treatment being administered.

For the disclosed compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

### IX. Subjects to be Treated

In certain embodiments, the patients to be treated according to the methods of the invention suffer from or are diagnosed with episodic migraine. Episodic migraine is diagnosed when patients with a history of migraine (e.g., at least five lifetime attacks of migraine headache) have 14 or fewer migraine headache days as defined herein per month. In some embodiments, patients having, suffering from, or diagnosed with episodic migraine have at least four, but less than 15 migraine headache days per month on average. In related embodiments, patients having, suffering from, or diagnosed with episodic migraine have fewer than 15 headache days per month on average. As used herein, a “headache day” is any calendar day in which the patient experiences a migraine headache as defined herein or any headache that lasts greater than 30 minutes or requires acute headache treatment. In some embodiments, the patient may be classified as having or suffering from high-frequency episodic migraine. High-frequency episodic migraine can be characterized by 8 to 14 migraine headache days per month. In other embodiments, the patient may be classified as having or suffering from low-frequency episodic migraine. Low-frequency episodic migraine can be characterized by fewer than 8 migraine headache days per month.

In some embodiments, the patients to be treated according to the methods of the invention suffer from or are diagnosed with chronic migraine. Chronic migraine is diagnosed when migraine patients (i.e., patients with at least five lifetime attacks of migraine headache) have 15 or more headache days per month, and at least 8 of the headache days are migraine headache days. In some embodiments, patients having, suffering from, or diagnosed with chronic migraine have 15 or more migraine headache days per month on average. In certain embodiments of the methods described herein, administration of NL-17 prevents, reduces, or delays the progression of episodic migraine in the patient to chronic migraine.

In certain embodiments of the methods described herein, the patient is treatment-naïve. In one embodiment, a patient is treatment-naïve if the patient has not previously received treatment for migraine headaches. In another embodiment, the patient is treatment-naïve if the patient was not administered a therapeutic agent for the treatment of migraine headaches. In some embodiments, a patient is treatment-naïve if the patient has not previously received prophylactic therapy for migraine headaches. For instance, in certain embodiments, a treatment-naïve patient has not received prior therapy or has not been administered a therapeutic agent for the prophylactic treatment of episodic migraine. In certain other embodiments, a treatment-naïve patient has not received prior therapy or has not been administered a therapeutic agent for the prophylactic treatment of chronic migraine.

In some embodiments of the methods described herein, the patient has failed or is intolerant to at least one other migraine headache prophylactic therapy. For example, in one particular embodiment, the patient has failed to respond to prior therapy with at least one migraine headache prophylactic agent. As used herein, “failure to respond” or “treatment failure” refers to the lack of efficacy of the prophylactic agent in reducing the frequency, duration, and/or severity of migraine headache in the patient following a standard therapeutic regimen of the agent. For instance, in one embodiment, a patient who has failed prior treatment with a migraine prophylactic agent is a patient who experienced the same or a greater number of monthly migraine headache days following administration of the migraine prophylactic agent as compared to the number of monthly migraine headache days prior to treatment with the agent. In another embodiment, a patient who has failed prior treatment with a migraine prophylactic agent is a patient who experienced the same or a greater number of monthly acute migraine-specific medication treatment days following administration of the migraine prophylactic agent as compared to the number of monthly acute migraine-specific medication treatment days prior to treatment with the agent. In yet another embodiment, a patient who has failed prior treatment with a migraine prophylactic agent is a patient who experienced the same or a greater number of migraine attacks following administration of the migraine prophylactic agent as compared to the number of migraine attacks prior to treatment with the agent. In still another embodiment, a patient who has failed prior treatment with a migraine prophylactic agent is a patient who experienced the same level or a greater level of physical impairment (e.g., mean monthly days with physical impairment) as measured by the MPFID following administration of the migraine prophylactic agent as compared to the level of physical impairment prior to treatment with the agent.

Failure to respond to prior treatment with a migraine prophylactic agent can also include inability to tolerate the migraine prophylactic agent. For example, in some embodiments, a patient who has failed prior treatment with a migraine prophylactic agent is a patient who cannot tolerate the side effects associated with the agent. In such embodiments, the side effects associated with the agent may exacerbate or may be incompatible with another medical condition that the patient has. By way of illustration, migraine prophylactic agents having a side effect of teratogenicity would be contraindicated in a pregnant patient. In certain embodiments, a patient who has failed prior treatment with a migraine prophylactic agent is a patient who discontinues treatment with the migraine prophylactic agent due to associated side effects. In these and other embodiments, a patient who has failed prior treatment with a migraine prophylactic agent is one who elects to stop treatment, alter the treatment regimen, or switch to a different prophylactic agent because the impact of the side effects outweighs the therapeutic benefit of the migraine prophylactic agent.

Migraine prophylactic agents include, but are not limited to, beta-blockers (e.g., propranolol, timolol, atenolol, metoprolol, and nadolol), antiepileptics (e.g. divalproex, sodium valproate, valproic acid, topiramate, and gabapentin), tricyclic antidepressants (e.g., amitriptyline, nortriptyline, doxepin, and fluoxetine), and botulinum toxin type A. Thus, in certain embodiments, the patients treated according to the methods of the invention have failed or are intolerant to one or more of these migraine prophylactic agents. In some embodiments, the patient has failed or is intolerant to treatment with at least two migraine prophylactic agents. In other embodiments, the patient has failed or is intolerant to treatment with at least three migraine prophylactic agents. In certain embodiments, the patient has failed or is intolerant to treatment with one or more agents selected from propranolol, timolol, divalproex, valproic acid, topiramate, amitriptyline, or botulinum toxin type A. In one particular embodiment, the patient has failed or is intolerant to treatment with topiramate. In another particular embodiment, the patient has failed or is intolerant to treatment with propranolol. In yet another particular embodiment, the patient has failed or is intolerant to treatment with amitriptyline.

In some embodiments, the patient has failed or is intolerant to treatment with two different classes of migraine prophylactic agents. For instance, in one embodiment, the patient may have failed or is intolerant to treatment with an antiepileptic (e.g., topiramate) and a beta-blocker (e.g., propranolol). In another embodiment, the patient may have failed or is intolerant to treatment with an antiepileptic (e.g., topiramate) and an antidepressant (e.g., amitriptyline). In still another embodiment, the patient may have failed or is intolerant to treatment with a beta-blocker (e.g., propranolol) and an antidepressant (e.g., amitriptyline). In certain embodiments, the patient has failed or is intolerant to treatment with three different classes of migraine prophylactic agents. In such embodiments, the patient has failed or is intolerant to treatment with an antiepileptic (e.g., topiramate), a beta-blocker (e.g., propranolol), and an antidepressant (e.g., amitriptyline).

The methods described herein are also applicable to other types of headache disorders, such as tension-type headaches, cluster headaches, hemiplegic migraine, and retinal migraine. Accordingly, the present invention also provides methods for treating, including prophylactically treating, or preventing any of the aforementioned headache disorders by administering an NL-17 to a patient in need thereof with any of the dosage regimens described herein.

The following examples are provided to illustrate the present disclosure, but are not intended to be limiting. The chemicals and physical parameters are presented as typical reagents or parameters, and various substitutions or modifications may be made in view of this disclosure by one of skill in the art without departing from the principle and spirit of the present invention.

### EXAMPLES

### Example 1: Preventive Migraine Treatment

### Materials and Methods

Animals: Female C57 (Charles River, USA) mice were used. Mice were 8-10 weeks of age upon receipt and allowed to acclimate for 3 days before use. Mice were housed in groups of 4 per cage, on a 12 hr light cycle with food and water ad libitum. Lights were turned on at 6 AM and turned off at 6 PM. For all assays, investigators were blinded to drug treatment, and animals were randomized to each treatment group prior to commencement of experiments. For each assay, mice were brought to the experimental room 1 hr prior to use. Animal procedures were approved by an IACUC and were performed in accordance with NIH standards and ARRIVE guidelines.

Reagents and antibodies: CGRP was from Sigma Aldrich (Cat #C0292) and used to induced migraine-like behavior (Rea, B. J., et al., Pain, 2018. 159(11): p. 2306-2317). NL-17 was prepared as described previously (Allen, D., et al. Sci. Rep. 10, 1-12 (2020); Allen, D., et al. Sci Rep 13:11778. (2023); Pavlik, B. J., et al. Sci. Rep. 6, 1-10 (2016)). Plasmid encoded constructs for C2I and C2II-C1 were expressed in E. coli BL21. E. coli was cultured in LB medium supplemented with ampicillin (100 g/mL) at 37° C. and were induced at an optical density of ~0.6 at 600 nm wavelength with 0.5 mM Isopropyl β-D-1-thiogalactopyranoside (IPTG). A French pressure cell was used to lyse the cell paste at 690 bar. Glutathione resin (Genscript) was used for affinity purification. GST fusion tags were removed using thrombin (Thermo Fisher). C2II-C1 was further activated using trypsin by incubation at 37° C. for 30 min at a 1:5 enzyme to substrate ratio as previously described (Barth, H. et al., 2000). A custom-made polyclonal antibody targeting the NL-17 enzymatic component C2I, was prepared in rabbits, as described in U.S. Pat. No. 10,633,643. The other antibodies used were goat anti-mouse IgG (H/L) polyclonal antibody (BioRad, Cat no. STAR207P) and goat anti-rabbit IgG (H/L): HRP (BioRad, Cat no. STAR124P). Thermo Scientific Pierce TMB substrate was used for ELISAs.

Drug administration: CGRP or vehicle (PBS) for migraine sensitization was administered by injection (IP) at 0.1 g/g bodyweight with a 30 g×0.5 needle. Mice were allowed to recover for 30 min after CGRP administration and before subsequent use in assays. NL-17 was applied in a subcutaneous location at the back thoracic region (base of trigeminal neuron) (Navratilova, E., et al., Cephalalgia, 42(11-12): p. 1194-1206 (2022)) and subcutaneous space in each check (Kosaras, B., et al., J Comp Neurol, 515(3): p. 331-48 (2009)) either 2 hr prior to CGRP sensitization (preventative migraine assay) or 15 min after (acute/reactive migraine assay). NL-17 dose was 0.5 pmol/g bodyweight divided into the three separate injections.

Light aversion, motility and grimace assays: Assays were performed as described (Navratilova, E., et al., 2022, Langford, D. J., et al., Nature Methods, 7(6): p. 447-449 (2010)) and video recorded. The light/dark data was performed in a standard light/dark chamber. Mice were pre-exposed to the chamber once for 30 minutes as a baseline measurement, then tested by exposure to bright light at 10,000 lx. The duration of time spent in the bright light chamber versus the dark chamber was collected over the 30 min assay, binned into sequential 5 min intervals and summed over the assay period. The assay depended on the exploratory drive of the animals and was limited to 30 min since mice tend to stop exploring if left in the chamber for longer times (Navratilova, E., et al., 2022, Langford, D. J., et al., 2010). Grimace scale scoring was conducted as described (Rea, B. J., et al., 2018, Navratilova, E., et al., 2022, Langford, D. J., et al., 2010).

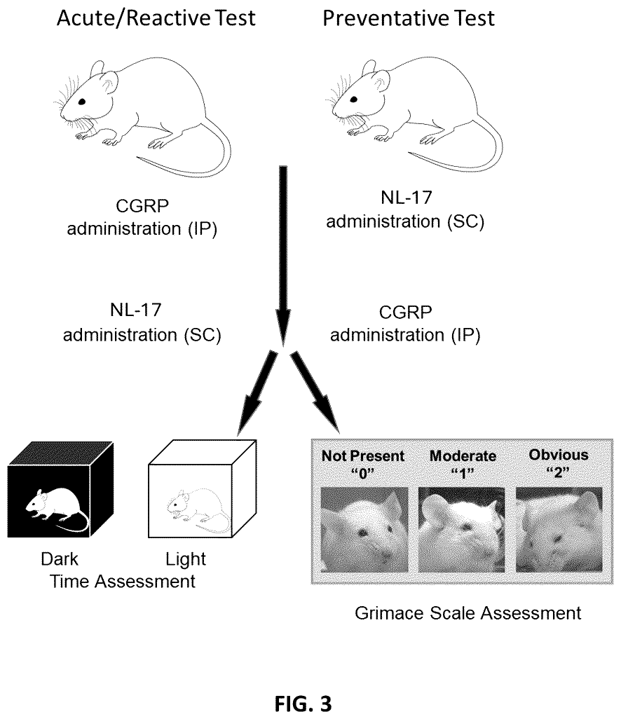

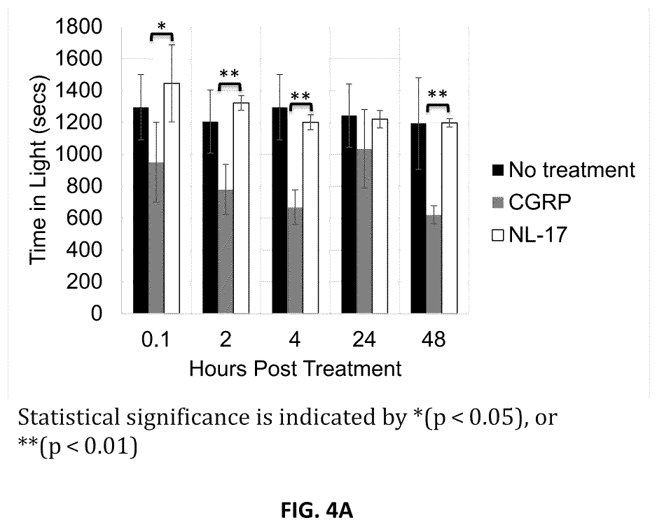

Experimental Design: A preclinical animal model (mouse) was used for migraine-like pain and behavior to study the benefit (efficacy) of NL-17 for migraine treatment (FIG. 3). This model involves the use of a peptide called CGRP known in humans to induce most types of migraines and in mice to induce migraine-like behavior. The experimental design involved either initial treatment with NL-17 followed by CGRP treatment (migraine prevention assay) or the reverse, CGRP treatment followed by NL-17 treatment (acute migraine treatment assay). Migraine-like behavior was measured in two ways, the amount of sensitivity to light or facial grimacing.

### Results

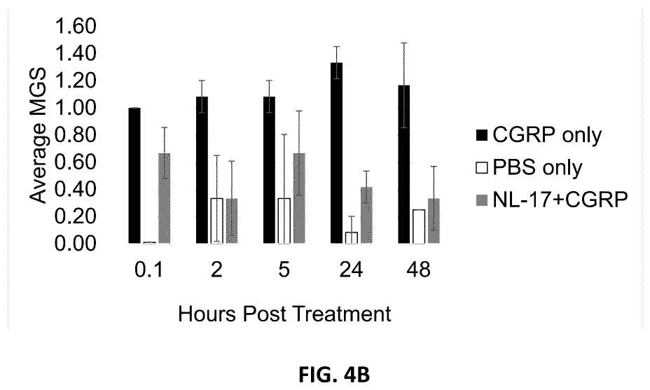

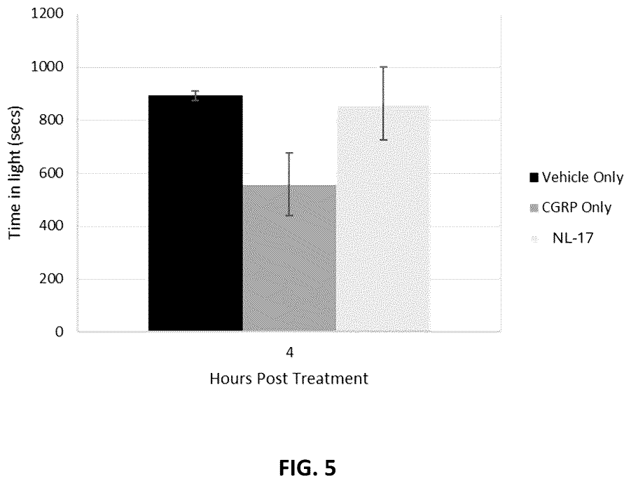

NL-17 was highly effective at preventing migraine-like behavior (FIGS. 4A-4B). NL-17 was administered before initial CGRP treatment and behavior was monitored from minutes to hours to days thereafter. Because CGRP in this model lasts only 5 hr, it was readministered 30 min before the 24 and 48 hr behavior measurements. NL-17 was not readministered. CGRP treated and CGRP and NL-17 treated animals were compared to untreated (vehicle only) controls. Relative to untreated mice, CGRP treated mice exhibited both light sensitivity (FIG. 4A) and increased grimace scores (FIG. 4B). Throughout the 48 time period, a single dose of NL-17 suppressed all CGRP-induced light sensitivity (FIG. 4A). NL-17 also suppressed grimacing at the 48 hr time point (FIG. 4B).

### Example 2: Acute (Also Called Reactive) Migraine Treatment

NL-17 was also effective at treating acute migraine like behavior. CGRP was administered 30 min before either vehicle or NL-17 and animal behavior as light sensitivity was monitored after four hours. At this time interval CGRP increased light sensitivity relative to vehicle only while NL-17 overcame the induced behavior caused by CGRP.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A composition comprising:
a) a target cell binding unit;
b) a pore-forming unit,
c) a rigid synthetic linker linking the target cell binding unit and the pore-forming unit, and
d) a payload unit comprising the agent, wherein said payload unit binds non-covalently to a pore formed by the pore-forming unit upon activation and oligomerization of the pore-forming unit, wherein the payload unit has at least 90% sequence identity to the amino acid sequence of SEQ ID NO: 2;
wherein the pore-forming unit is a polypeptide derived from a first AB type of non-neurotoxic binary toxin, and wherein the target cell binding unit is a polypeptide derived from a second AB type of toxin, said second AB type being different from the first AB type, wherein the target cell binding unit binds to a neural cell,
wherein the target cell binding unit and the pore-forming unit are covalently linked through the rigid synthetic linker to form a polypeptide.

2. The composition of claim 1, wherein the pore-forming unit is a polypeptide or a polypeptide oligomer derived from the heavy chain of the pore-forming unit of Clostridium botulinum toxin C2.

3. The composition of claim 1, wherein the pore-forming unit comprises a native or modified heavy chain binding domain derived from a toxin other than C. botulinum toxin C2.

4. The composition of claim 1, wherein the polypeptide comprising target cell binding unit, rigid synthetic linker, and pore-forming unit has at least 90% sequence identity to the amino acid sequence of SEQ ID NO: 1.

5. The composition of claim 1, wherein the payload unit is not covalently bound to the target cell binding unit or the pore-forming unit.

6. The composition of claim 1, wherein the composition is administered to the subject by subcutaneous injection, transdermal (microneedling patch), intradermal, intramuscular, intravenous, intraperitoneal, or intrathecal administration.

7. The composition of claim 1, wherein the composition is administered in combination with one or more antiepileptic agents, tricyclic antidepressants, beta-blockers, or migraine headache prophylactic agents, the migraine headache prophylactic agents selected from propranolol, timolol, divalproex, valproic acid, topiramate, amitriptyline, nicotinamide adenine dinucelotide (NAD+), anti-CGRP or anti-CGRP receptor antibodies, or the combination thereof.

8. The composition of claim 1, wherein the composition is adapted to deliver molecules into peripheral sensory neurons, and wherein the composition does not substantially affect motor neurons.

9. The composition of claim 1, wherein the composition interacts with receptor ganglioside GT1b.

10. A method for treating, preventing, or reducing the occurrence of headache disorders in a subject in need thereof comprising administering to the subject an effective amount of the composition of claim 1.

11. The method of claim 10, wherein the patient has or is diagnosed with a headache disorder selected from episodic migraine headaches, tension-type headaches, cluster headaches, hemiplegic migraine headaches, or retinal migraine headaches.

12. The method of claim 10, wherein the patient has or is diagnosed with chronic migraine.

13. The method of claim 10, wherein the patient has at least four, but fewer than migraine headache days per month.

14. The method of claim 10, wherein the patient has failed or is intolerant to other classes of migraine headache prophylactic agents.

15. A method for prophylactically treating headache disorders in a subject in need thereof comprising administering to the subject an effective amount of the composition of claim 1.

16. The method of claim 15, wherein the patient has or is diagnosed with a headache disorder selected from chronic migraines, episodic migraine headaches, tension-type headaches, cluster headaches, hemiplegic migraine headaches, or retinal migraine headaches.

17. The method of claim 15, wherein the pharmaceutical composition is administered in combination with one or more migraine headache prophylactic agents selected from propranolol, timolol, divalproex, valproic acid, topiramate, amitriptyline, nicotinamide adenine dinucelotide (NAD+), anti-CGRP or anti-CGRP receptor antibodies, or the combination thereof.

18. The method of claim 16, wherein the pharmaceutical composition is administered in combination with an antiepileptic, a tricyclic antidepressant, and a beta-blocker.

19. The method of claim 15, wherein the patient has failed or is intolerant to other classes of migraine headache prophylactic agents.


SEQUENCE LISTING (2 sequences)

SEQ ID NO: 1 - protein, 861 aa, synthetic construct
MANANRDTDRDGIPDEWEINGYTVMNQKAVAWDDKFAANGYKKYVSNPFKPCTANDPYTDFEKVSGQIDPSVSMVARDPMISAYPIVGVQMERLVVSKSETITGDSTKSMSKSTSHSSTNINTVGAEVSGSLQLAGGIFPVFSMSASANYSHTWQNTSTVDDTTGESFSQGLSINTAESAYINPNIRYYNTGTAPVYNVTPTTTIVIDKQSVATIKGQESLIGDYLNPGGTYPIIGEPPMALNTMDQFSSRLIPINYNQLKSIDNGGTVMLSTSQFTGNFAKYNSNGNLVTDGNNWGPYLGTIKSTTASLTLSLPDQTTQVAVVAPNFSDPEDKTPRLTLEQALVKAFRLEKKNGKFYFHGMEISANQKIQVFLDRNTNVDFENQLKNTANKDIMNCIIKRNMNILVKVITGSEPEPEPEPEPEPEPEPEPEPGNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYSTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 2 - protein, 431 aa, synthetic construct
MAIIKEPIDFINKPESEAKKWGKEEEKRWFTKLNNLEEVAVNQLKNKEYKTKIDNFSTDILFSSLTAIEIMKEDENQNLFDVERIREALLKNTLDRDAIGYVNFTPKELGINFSIRDVELDRDISDETLDKVRQQIINQEYTKFSFISLGLNDNSINESVPVIVKTRVPTTFDYGVLNDKETVSLLLNQGFSIIPESAIITTIKGKDYILIEGSLSQELDFYNKGSEAWGAENYGDYISKLSHEQLGALEGYLHSDYKAINSYLRNNRVPNNDELNKKIELISSALSVKPIPQTLIAYRRVDGIPFDLPSDFSFDKKENGEIIADKQKLNEFIDKWTGKEIENLSFSSTSLKSTPLSFSKSRFIFRLRLSEGTIGAFIYGFSGFQDEQEILLNKNSTFKIFRITPITSIINRVTKMTQVVIDAEVIQNKEI